(19) United States
(12) Patent Application Publication
(10) Pub. No.: US 2015/0011952 A9
(43) Pub. Date: Jan. 8, 2015

(54) Onlay subcutaneous injection port

(71) Applicant: Sidney P. Rohrscheib, Clinton, IL (US)

(72) Inventor: Sidney P. Rohrscheib, Clinton, IL (US)

(21) Appl. No.: 13/135,537

(22) Filed: Jul. 8, 2011

Related U.S. Application Data
(63) Continuation of application No. PCT/US09/00142, filed on Jan. 9, 2009.

(51) Int. Cl. A61M 5/00

(52) U.S. Cl. 604/288.02

(57) ABSTRACT

The device is an implantable subcutaneous injection port used for various purposes including, but not limited to, vascular access and laparoscopic adjustable gastric banding. This device, with its tubing route and flexible connectors offers a solution to the problem of tubing occlusion and loss of integrity due to kinking. This device exhibits an injection chamber disposed within a housing. A self sealing septum is disposed within an injection chamber aperture. A second aperture communicates with a tube that is designed to connect to a catheter or other device suitable to the surgical application. Here the second aperture exits directly beneath the onlay port reducing the possibility of tubing kink, needle puncture and malpositioning seen with only ports where the catheter exits from the side of the device. An alternate embodiment utilizes a u-joint and ball joint to maximize the flexibility of the connection between the onlay port and the exiting catheter.

### CROSS REFERENCE TO RELATED APPLICATIONS

This application is the National Stage of International Application No. PCT/US2009/000142, filed 9 Jan. 2009.

### TECHNICAL FIELD

Implantable subcutaneous injection ports have been used primarily for the purpose of vascular access. The devices are generally implanted beneath the skin of a patient near the upper chest. The injection port exhibits a silicon membrane overlying a chamber which is, in turn, connected to tubing and catheter usually entering the internal jugular or subclavian vein. The chamber is accessed by puncturing the overlying skin with a Huber point needle, then puncturing the self sealing silicon membrane of the port. This allows the repeated injection of medicines or the drawing of blood with low risk of infection at the same time preserving the integrity of the skin. Various injection ports are commercially available. By way of example is the injection port available under the brand name of Port-A-Cath® Implantable Venous Access Systems.

### DISCLOSURE OF THE INVENTION

The onlay access port exhibited here represents an advance in the design of subcutaneous medical access devices. This is especially true for applications where the access port is placed on the abdominal fascia, and where the tubing egress route is through the abdominal musculature and into the peritoneal cavity. Such an application is laparoscopic adjustable gastric banding. Ports currently used in laparoscopic adjustable gastric banding borrow their design from those traditionally used as venous access devices placed on the chest wall. Traditional venous access ports exhibit tubing exiting from the side of the port which is advantageous in these applications where a length of tubing will initially track more or less horizontally, parallel to the chest wall then entering a large vein. Venous access applications present minimal opportunity for the tubing to be routed at sharp angles and consequently less opportunity for kinking and resulting obstruction. Use of venous access ports in procedures such as laparoscopic adjustable gastric banding where it requires the routing of tubing through the abdominal fascia and into the peritoneal cavity instead of routing the tubing horizontally along the chest wall. This often requires the tubing to be routed at sharper angles. This results in the most common complications in laparoscopic adjustable gastric banding which are the occlusion of tubing due to kinking and the loss of integrity of the tubing wall by cracking due to angulation stresses. The onlay port described here shows an exit directly beneath and at the bottom of the onlay access port allowing direct vertical penetration of the abdominal fascia by the tubing which exits the port at right angles to the horizontal orientation of the onlay port.

This new design also reduces the possibility of puncturing the tubing during needle access to the port and reduces the possibility of port to become malpositioned or rotated due to the horizontal fixed tubing which eliminates all but one axis of freedom about which it can rotate. Additionally the onlay access port will be easier to place at surgery. Direct placement over a trochar site allow the tubing to be inserted through a smaller incision immediately below the only access port. This further enhances the stability of the port installation and reduces the need for fixation to the muscular fascia by suturing or other fixative technique. The only access port will be manufactured of titanium and silicone or other suitable materials that are inert and well tolerated by the body

Alternative embodiments of the port utilizing u-joint and ball joint connectors, will add additional flexibility in the catheter attachment allowing the patent greater freedom of movement with lessened risk of dislodging the access port.

### BRIEF DESCRIPTION OF THE DRAWINGS

FIG. 1 is a cross section view of the onlay access port.

FIG. 2 is a perspective view of the onlay access port components.

FIG. 3 is a perspective view of the onlay port bottom.

FIG. 4 is a perspective view of the only port embodiment showing joined halves of the injection chamber housing.

FIG. 5 is a perspective view of the injection chamber housing halves disengaged.

FIG. 6 is a map view of the injection chamber housing halves disengaged.

FIG. 7 is a cross section view of the onlay port show an angular catheter.

FIG. 8 is a cross section view of the onlay port with an angular catheter seat.

FIG. 9 is a cross section view showing the modified injection chamber housing bottom aperture.

FIG. 10 is a map view of first shield, second shield and second shield aperture.

FIG. 11 is a cross section view an alternative configuration of the shield, second shield and second shield aperture.

FIG. 12 is a cross section view of the u-joint.

FIG. 13 is a cross section view of the ball joint.

FIG. 14 is a cross section view of the ball joint with o-ring retainer is place.

Component List

| | |
|---|---|
| Self-sealing septum | 1 |
| Septum seat | 2 |
| Septum seat | 2A |
| Injection chamber sidewall | 3 |
| Injection chamber bottom | 4 |
| Fluid path | 5 |
| First chamber aperture | 5A |
| First housing aperture | 5B |
| Catheter seat | 6 |
| First catheter seat end | 7 |
| Second catheter seat end | 7A |
| Barbs | 7C |
| Sidewall lip | 8 |
| Injection chamber sidewall | 9 |
| Injection chamber housing bottom | 10 |
| Injection chamber housing bottom aperture | 10A |
| Shoe | 11 |
| Parabolic lip | 11A |
| Injection chamber | 12 |
| Catheter | 13 |
| Catheter end | 13A |
| Catheter end | 13B |
| Catheter wall | 13D |
| Catheter fluid path | 13C |
| Injection chamber housing | 14 |
| Barbs | 15 |
| First barb end | 15A |
| Second barb end | 15B |
| Secondary barbs | 15C |
| Chamber housing first half | 16 |
| Chamber housing second half | 17 |
| Second joining insert | 18 |
| Expanded lip | 18A |
| Joining pocket | 19 |
| Narrowed lip retainer | 19A |
| Second joining pocket | 20 |
| Joining surfaces | 20A |
| Joining insert | 21 |
| Angular catheter | 22 |
| Bottom aperture | 25 |
| First shield | 26 |
| Bearing support | 28 |
| Bearings | 29 |
| First shield aperture | 30 |
| Second shield | 32 |
| Second shield aperture | 32A |
| Bearing support aperture | 34 |
| U-joint | 35 |
| Upper u-joint component | 35A |
| Lower u-joint component | 36 |
| Fluidly sealed hinge | 36A |
| Ball housing | 37 |
| First ball housing end | 37A |
| Second ball housing end | 37B |
| Ball housing threads | 38 |
| Ball component | 39 |
| Channel | 40 |
| Ball joint assembly | 40A |
| Catheter seat | 41 |
| Ball catheter seat first end | 41A |
| Ball catheter seat second end | 41B |
| Ball catheter channel | 41C |
| First o-ring | 43A |
| O-ring retainer | 44 |
| First ball housing end aperture | 46 |
| Second ball housing aperture | 48 |

### BEST MODE OF CARRYING OUT THE INVENTION

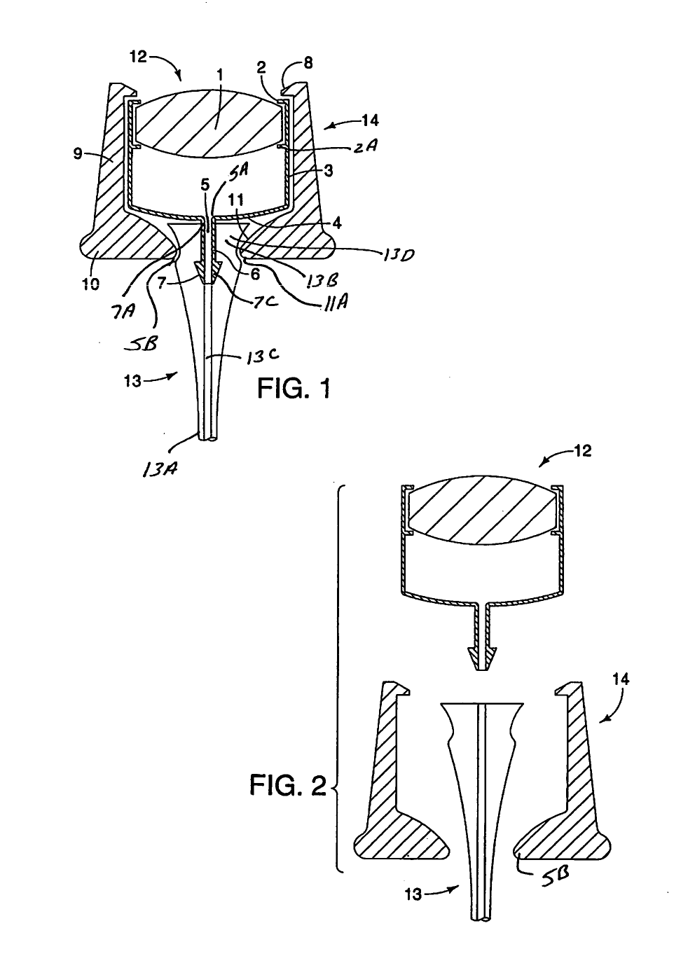

FIG. 1 shows the three major components of the access port, which are the injection chamber housing 14 with injection chamber 12 disposed within. Also partially disposed within the injection chamber housing is catheter 13. Catheter 13 exhibits a first catheter end 13A and a second catheter end 13B. Catheter 13 also exhibits a catheter fluid path 13C. Second catheter end 13B is disposed over catheter seat 6. Catheter seat 6 exhibits a first catheter seat end 7 and a second catheter seat end 7A. First catheter seat end 7 exhibits barbs 7C. Second catheter seat end 7A is attached to injection chamber bottom 4. Fluid path 5 extends through the center of catheter seat 6 and fluidly communicates with the injection chamber through first chamber aperture 5A in injection chamber bottom 4. Catheter 13 is disposed over catheter seat 6. The fluid path 5 of catheter seat 6 is fluidly communicates with the catheter fluid path 13C of catheter 13. Injection chamber housing 14 is annular in shape and is composed of injection chamber sidewall 9, injection chamber housing bottom 10, and sidewall lip 8. Injection chamber housing 14 exhibits first housing aperture 5B which penetrates injection chamber housing bottom 10 through first housing aperture 5B. First housing aperture 5B exhibits shoe 11. Shoe 11 exhibits a parabolic lip 11A. The parabolic lip 11A will of course be annular in shape following the path of the first housing aperture 5B of injection chamber housing bottom 10. The parabolic lip 11A of shoe 11 applies pressure to Catheter wall 13D which are compressed between parabolic lip 11A and catheter seat 6. The compression thus allows catheter 13 to fluidly seal against catheter seat 6 and further to be retained upon catheter seat 6 and within injection chamber housing 14. Injection chamber sidewall 3 exhibits septum seat 2 and septum seat 2A. Self-sealing septum 1 is mounted within the injection chamber and sealed against the injection chamber sidewalls 3 by the septum seat 2 and septum seat 2A. Septum seat 2 and septum seat 2A may retain self-sealing septum 1 by crimping or providing a stable seat for fixing the self-sealing septum with adhesive.

FIG. 2 shows the three major components of the onlay access port disassembled but in a configuration prior to the assembly of the port. This figure shows injection chamber 12 above catheter 13 which is passed through first housing aperture 5B of the injection chamber housing bottom 10. This exhibits one of the several advantages of this improved port design. A number of circumstances can be envisioned in which it would be advantageous to have the capacity to assemble or disassemble the port. These situations might include a desire to reposition the port, or a need to repair the tubing proximate to the base of the port.

Turn now to FIG. 3, an alternative embodiment of the onlay port is seen where injection housing bottom 10 exhibits a plurality of barbs 15. Catheter 13 is designed to be inserted through a trochar site in the fascia. Injection chamber housing bottom 10 then rests on the fascia. In order to secure the onlay port to the fascia, the barbs having a first barb end 15A and a second barb end 15B then rests on the fascia. First barb end 15A is attached to injection chamber housing bottom 10. Second barb end 15B is free floating. When the injection chamber housing 14 is rotated, the second barb end 15B then penetrates the fascia. The second barb end 15B may also exhibit secondary barbs 15C allowing the barbs to be held within the fascia.

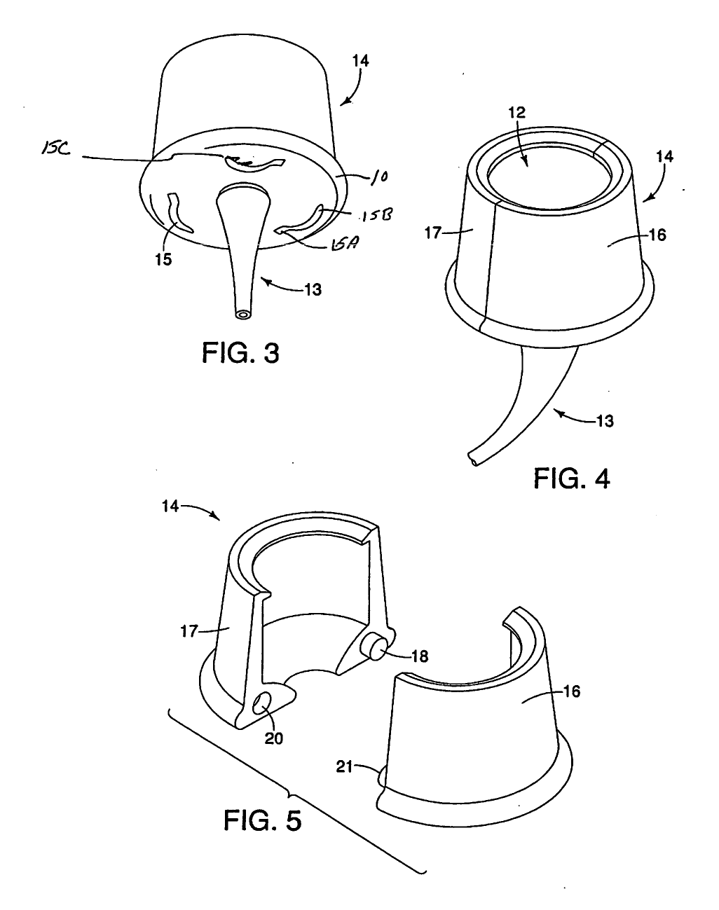

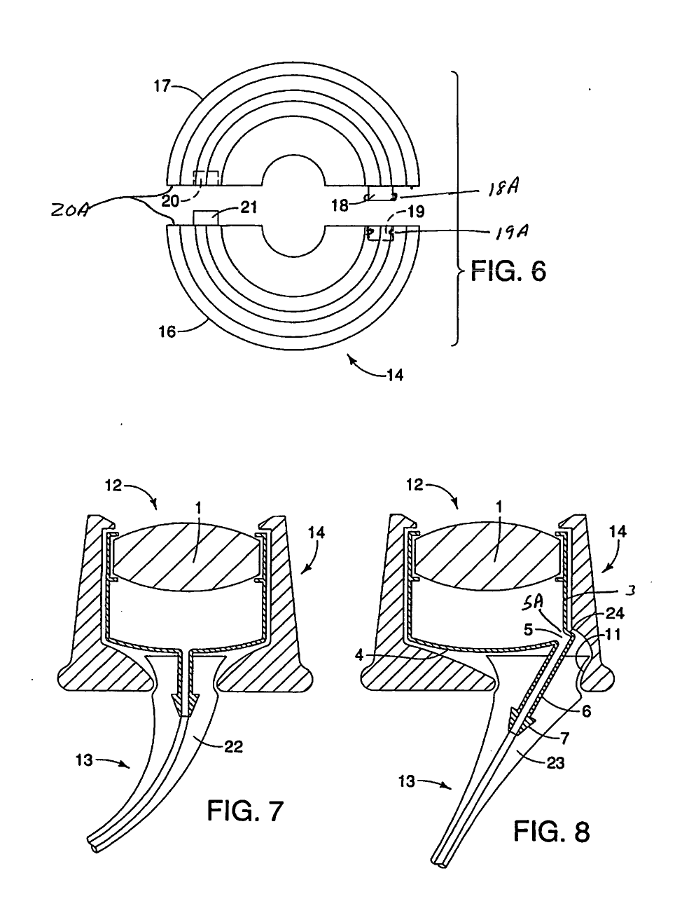

Another embodiment of the onlay port is exhibited in FIG. 4, where injection chamber housing 14 is shown having chamber housing first half 16 and chamber housing second half 17. The chamber housing halves are shown assembled in FIG. 4. FIG. 5 shows the chamber housing halves separated. FIG. 6 shows chamber housing first half exhibiting joining pocket 19 and joining insert 21. Chamber housing second half 17 exhibits second joining insert 18 and second joining pocket 20. When chamber housing first half 16 is joined to chamber housing second half 17, second joining insert 18 is disposed within joining pocket 19 and joining insert 21 is disposed within second joining pocket 20. A plurality of joining inserts and corresponding joining pockets may be exhibited up and down the joining surfaces 20A of chamber housing first half 16 and chamber housing second half 17.

The joining pockets and the joining inserts may be configured in such a way that when joining insert is disposed within the joining pocket, it is retained therein by means of an expanded lip 18A on second joining insert 18 which is retained by a narrowed lip retainer 19A in joining pocket 19. Alternatively joining pockets and joining inserts could be smooth-walled and would be joined by an appropriate adhesive.

As mentioned earlier, the appropriate routing of catheter 13 is to avoid kinking which is a significant complication in the use of these devices in laproscopic adjustable gastric banding. FIG. 7 exhibits angular catheter 22 where in the catheter is molded in an angular configuration. If the surgeon determines a particular orientation of the catheter is appropriate, the angular catheter 22 can then be oriented in the appropriate direction either after or prior to onlay port component assembly.

FIG. 8 exhibits yet another embodiment of the onlay port. First chamber aperture 5A appears where injection chamber sidewall 3 meets injection chamber bottom 4. This places the first chamber aperture 5A well away from an injection needle. The catheter seat 6 then extends from injection chamber bottom 4 at an angle thus creating the necessary angular routing should the surgeon determine such an angular routing is desirable.

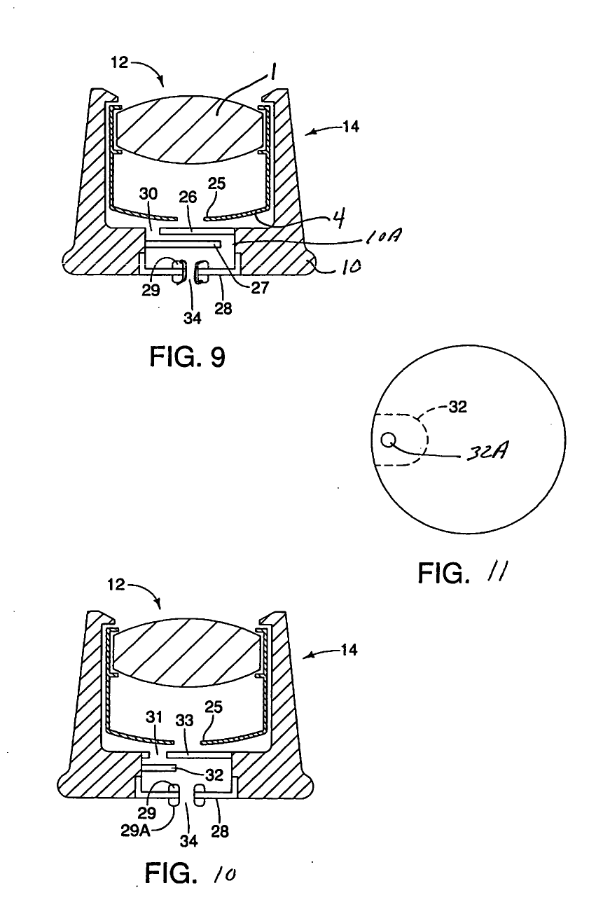

FIG. 9 shows an alternative embodiment of the onlay port wherein the catheter seat 6 is removed from injection chamber 14 allowing injection chamber bottom 4 to exhibit a centrally located bottom aperture 25. Immediately under the centrally located bottom aperture 25 is first shield 26. First shield 26 is fixed to the internal portion of the injection chamber housing bottom 10 and extends across injection chamber housing bottom aperture 10A. This is designed to prevent the needle from penetrating the self-sealing septum septum 1 and continuing on to penetrate and to potentially compromise the integrity of a catheter. However first shield aperture 30 allows fluids to pass through and around second aperture shield 26. First housing aperture 10A is also modified by the addition of bearing support 28. Bearing support 28 is annular in nature extending around the circumference of first housing aperture 10A and exhibits bearing support aperture 34. Extending through baring support aperture 34 are bearings 29.

Injection chamber bottom aperture 25 need not be centrally located but may be located in any position such that fluid may pass from the injection chamber 12. It should also be noted that the first shield aperture 30 need not be confined to any particular location on first shield 26. FIG. 10 shows an alternate location for first shield aperture 30. Second shield 32 may then be significantly reduced in size and need cover only the first shield aperture 30 as can be seen in FIG. 11 where second shield aperture 32A is greatly reduced in size.

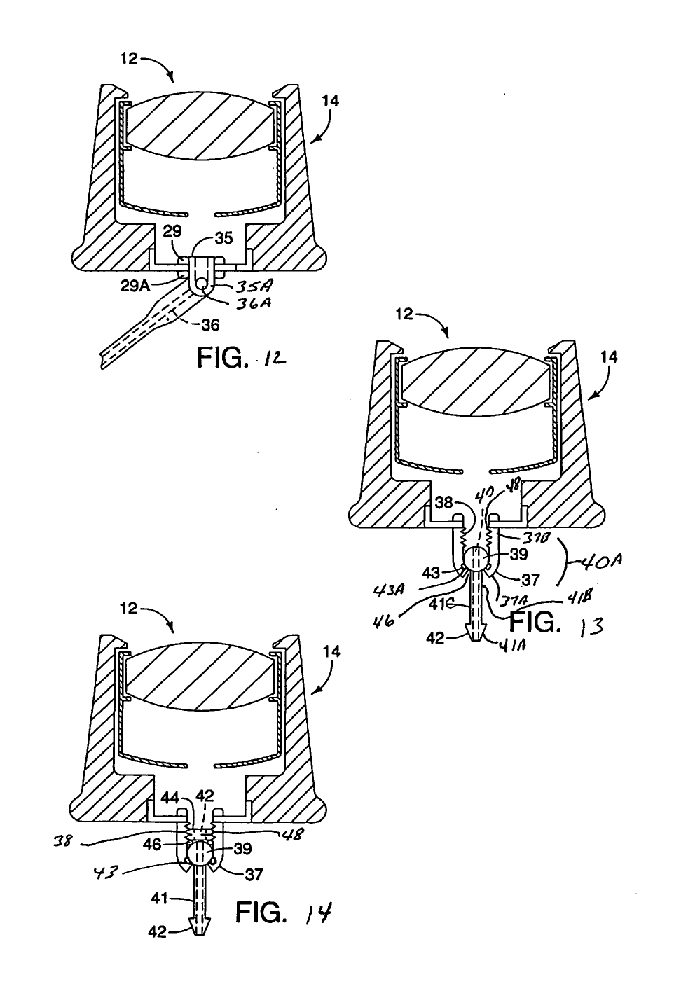

FIG. 12 shows u-joint 35 inserted through bearings 29. U-joint 35 exhibits an upper u-joint component 35A and a lower u-joint component 36. Upper u-joint component 35A may rotate 360 degrees upon bearings 29. Lower u-joint component 36 is attached through a fluidly sealed hinge 36A to upper u-joint component 35A. Lower u-joint component 36 may swing through an arc of 180 degrees. This, in combination with the ability for upper u-joint component 35 to rotate within bearings 29, allows positioning catheter 13 and any conceivable angle.

FIG. 13 shows ball joint assembly 40A disposed within bearing support aperture 34. Ball joint assembly 40A is composed of ball housing 37 which exhibits internal annular o-ring seat 43. Disposed within o-ring seat 43 is o-ring 43A. Ball housing 37 exhibits first ball housing end 37A and second ball housing end 37B. Second ball housing end 37B is mounted to bearing support 28 and fluidly communicates with bearing support aperture 34. First ball housing end 37A is rolled inward such that first ball housing end aperture 46 is smaller in diameter than the second ball housing aperture 48. Ball component 39 is composed of a sphere of a diameter allowing the ball component 39 to be disposed within ball housing 37 but of such a diameter that first ball housing end aperture 46 retains the ball component 39 within ball housing 37. Ball component 39 exhibits channel 40 allowing fluid flow through. Ball component 39 is attached to ball catheter seat 41 which exhibits ball catheter seat first end 41A, ball catheter seat second end 41B and ball catheter channel 41C. Ball catheter seat second end 41B ball component 39 and ball catheter channel 41C fluidly communicates with channel 40. When ball component 39 is in place at first ball housing end 37A, it rotates against first o-ring 43A providing a fluid tight seal. Ball component 39 may rotate within ball housing again allowing a significant range of angles that catheter 13 may adopt.

FIG. 14 shows ball housing 37 with the addition of o-ring retainer 44. O-ring retainer 44 exhibits retainer threads 48 on its outer portions. It can therefore be disposed within the ball housing threads 38. O-ring retainer 44 then depresses second o-ring 46 against top of ball component 39 maintaining a fluid tight seal.

### INDUSTRIAL APPLICABILITY

The invention is applicable to laparoscopic adjustable gastric banding surgery or any surgical procedure where the implantation of an access port with the characteristics of the above described invention would be desirable.

What is claimed is:

1. An onlay access port comprising:
a. an injection chamber housing having a first housing aperture and an opposing second housing aperture,
b. an injection chamber disposed within said injection chamber housing, having a first chamber aperture and an opposing second chamber aperture,
c. a self sealing septum sealably disposed within said second chamber aperture,
d. a catheter seat in fluid communication with said first chamber aperture,
e. a catheter having a first catheter end and a second catheter end, said second catheter end in fluid communication with said catheter seat, said catheter extending through said first housing aperture parallel with the longitudinal axis of said onlay port.

2. The onlay access port of claim 1 wherein said injection chamber further comprises:
a. injection chamber sidewalls,
b. injection chamber bottom connected to said injection chamber sidewall, said injection chamber bottom containing said first chamber aperture,
c. septum seats mounted to said injection chamber sidewalls whereby said self sealing septum is retained.

3. The onlay access port of claim 1 wherein said injection chamber housing further comprises:
a. injection chamber housing sidewalls,
b. injection chamber housing bottom connected to said injection chamber sidewalls, said injection chamber housing bottom containing said first housing aperture.
c. sidewall lip mounted on said injection chamber sidewalls whereby by said injection chamber is retained within said injection chamber housing,
d. said first housing aperture further comprising a shoe having a parabolic lip within the internal circumference of said first housing aperture whereby pressure may be applied to said second catheter end depressing said second catheter end against said catheter seat.

4. The onlay access port of claim 1 wherein said injection chamber housing, said injection chamber and said catheter may be assembled and disassembled.

5. The onlay access port of claim 1 wherein said injection chamber housing bottom further comprises a plurality of barbs, said barbs having a first barb end and a second barb end said first barb end mounted to said injection chamber housing bottom.

6. The onlay access port of claim 1 wherein said injection chamber housing further comprises a first injection housing half and a second injection housing half said injection housing halves capable of being joined to retain said injection chamber and wherein said first injection housing half has a first injection housing joining surface and wherein said second injection housing half has a second injection housing half joining surface said first injection housing half joining surface communicates with said second injection housing half joining surface.

7. The onlay access port of claim 6 further wherein said first injection housing half joining surface exhibits a plurality of joining pockets and wherein said second injection housing half joining surface exhibits a plurality of joining inserts said joining inserts capable of being disposed within said joining pockets whereby injection chamber housing halves are joined.

8. The onlay access port of claim 1 wherein said catheter is angularly molded.

9. The onlay access port of claim 1 wherein said catheter seat is angularly disposed from the injection chamber bottom.

10. An onlay access port comprising:
a. an injection chamber housing having a first housing aperture and an opposing second housing aperture,
b. an injection chamber disposed within said injection chamber housing, having a first chamber aperture and an opposing second chamber aperture,
c. a self sealing septum sealably disposed within said second chamber aperture,
d. a catheter seat rotatably joined and in fluid communication with said first chamber aperture
e. a catheter fluidly connected to said catheter seat.

11. The onlay access port of claim 10 where in said first housing aperture further comprises:
a. a first chamber aperture wall,
b. a bearing support connected to said first chamber aperture wall said bearing support having a bearing aperture,
c. a first shield having a first shield aperture, said first shield aperture connected to said first chamber wall at a point above said bearing support,
d. a second shield having a second shield aperture said second shield connected to said first chamber wall at a point above said bearing support and below said first shield wherein said first shield aperture and said second shield aperture are not opposing
e. a catheter seat rotatably and fluidly connected to said bearing aperture.
f. a cathether fluidly connected to said catheter seat.

12. The onlay access port of claim 11 further comprising:
a. a bearing array rotatably disposed within said bearing aperture.

13. The onlay access port of claim 12 wherein said catheter seat further comprises:
a. a u-joint assembly having,
b. an upper u-joint component rotatably and fluidly connected to said bearing assembly,
c. a lower u-joint component hingedly and fluidly connected to said upper u-joint component, said lower u-joint component connected to said catheter.

14. The onlay access port of claim 11 wherein said catheter seat further comprises;
a. a ball housing,
b. a ball rotatably disposed within said ball housing said ball having a channel therethrough fluidly communicating with said injection chamber,
c. a ball retainer whereby said ball is sealably retained within said ball housing.

15. The onlay access port of claim 14 wherein said ball housing and ball are self sealing when in communication.

16. The onlay access port of claim 14 wherein said ball housing further comprises:
a. a ball housing first end having a ball housing first aperture,
b. a ball housing second end having a ball housing second aperture,
c. said ball housing first end fluidly connected to said bearing aperture and of such a diameter that said ball may be disposed within said ball housing,
d. said ball housing second aperture of such a diameter smaller than said ball whereby said ball is retained in said ball housing.

17. The onlay access port of claim 14 wherein said ball housing further comprises:
a. an o-ring seat,
b. a first o-ring disposed within said o-ring seat,
c. ball housing threads internally disposed within ball housing,
d. a second o-ring disposed on said ball,
e. o-ring retainer threadedly disposed within said ball housing with whereby second o-ring is sealed against said ball.

18. The onlay access port of claim 2, wherein said first chamber aperture may be disposed in a plurality of locations on injection chamber bottom.

19. The only access port of claim 18 wherein said injection chamber aperture is located at the intersection of injection chamber sidewall and injection chamber bottom and where in said catheter seat is disposed angularly in relation to said injection chamber housing bottom.
